(19) United States
(12) Patent Application Publication
(10) Pub. No.: US 2005/0064431 A1
(43) Pub. Date: Mar. 24, 2005

(54) Biological microarray comprising polymer particles and method of use

(75) Inventors: Jeffrey W. Leon, Rochester, NY (US); Tiecheng A. Qiao, Webster, NY (US); Christine J. Landry-Coltrain, Fairport, NY (US)

Correspondence Address: Paul A. Leipold;Eastman Kodak Company, Patent Legal Staff, 343 State Street, Rochester, NY, 14650-2201, US

(73) Assignee: Eastman Kodak Company

(21) Appl. No.: 10/658,438

(22) Filed: Sep. 9, 2003

(51) Int. Cl. C12Q 1/68; C07H 21/04; C12M 1/34

(52) U.S. Cl. 435/6; 435/287.2; 536/24.3

(57) ABSTRACT

The present invention relates to a microarray comprising a support having attached to a surface thereof at least one porous layer, wherein the porous layer comprises a hydrophilic binder and polymer particles. The present invention also relates to a method of using a microarray comprising providing a microarray comprising a support having attached to a surface thereof at least one porous layer, wherein the porous layer comprises a hydrophilic binder and polymer particles; contacting the microarray with biological targets labeled with optical emission tag; and measuring the signals from the optical emission tag.

### CROSS REFERENCE TO RELATED APPLICATIONS

Reference is made to commonly assigned, co-pending U.S. patent application Ser. No. ______ by Leon et al. (Docket 85487) filed of even date herewith entitled “Stabilized Polymer Beads And Method Of Preparation”, the disclosure of which are incorporated herein.

### FIELD OF THE INVENTION

The present invention relates to a biological microarray of polymer particles and method of use therefor.

### BACKGROUND OF THE INVENTION

The completion of the Human Genome project spurred the rapid growth of a new interdisciplinary field of proteomics, which includes identification and characterization of complete sets of proteins encoded by the genome, the synthesis of proteins, post-translational modifications, as well as detailed mapping of protein interaction at the cellular regulation level.

While 2-dimensional gel electrophoresis in combination with mass spectrometry still remains the dominant technology in proteomics study, the successful implantation and application of deoxyribonucleic acid (DNA) microarray technology to gene profiling and gene discovery have prompted scientists to develop protein microarray technology and apply microchip based protein assays to the field of proteomics. For example, in WO 00/04382 and WO 00/04389, a method of fabricating protein microarrays is disclosed. A key element in the disclosure is a support consisting of a solid support coated with a monolayer of thin organic film on which protein or a protein capture agent can be immobilized.

Nitrocellulose membrane was widely used as a protein blotting support in Western blotting and Enzyme Linked Immunosorbent Assay (ELISA). In WO 01/40312 and WO 01/40803, antibodies are spotted onto a nitrocellulose membrane using a gridding robot device. Such spotted antibody microarrays on a nitrocellulose membrane support have been shown to be useful in analyzing protein mixture in a large parallel manner.

WO 98/29736 describes an antibody microarray with an antibody immobilized onto a N-hydroxysuccinimidyl ester modified glass support. In U.S. Pat. No. 5,981,734 and WO 95/04594, a polyacrylamide based hydrogel support technology is described for the fabrication of DNA microarrays. More recently, in Anal. Biochem. (2000) 278, 123-131, the same hydrogel technology was further demonstrated as useful as a support for the immobilization of proteins in making protein microarrays.

The common feature among these different approaches is the requirement of a solid support that allows covalent or non-covalent attachment of a protein or a protein capture agent on the surface of the support. In DNA microarray technology, a variety of surfaces have been prepared for the deposition of pre-synthesized oligos and polymerase chain reaction prepared cDNA probes. For example, in EP 1 106 603 A2, a method of preparing vinylsulfonyl reactive groups on the surface to manufacture DNA chips is disclosed. Even though the invention is useful in preparing DNA chips, it is not suitable for protein microarray applications. Unlike DNA, proteins tend to bind to surfaces in a non-specific manner and, in doing so, lose their biological activity. Thus, the attributes for a protein microarray support are different from those for a DNA microarray support in that the protein microarray support must not only provide surface functionalities that are capable of interacting with protein capture agents, but must also resist non-specific protein binding to areas where no protein capture agents have been deposited.

U.S. application Ser. No. 10/020,747 describes a low cost method of making protein microarray supports using a gelatin coating to create a reactive surface for immobilization of protein capture agents. While the gelatin modified surface effectively eliminates non-specific protein binding, the number of reactive sites on the surface are limited by the intrinsic functional groups in gelatin and the type of chemical agents (A-L-B) employed.

Since the number of reactive sites on the surface directly determines the ultimate signal detection limit, it is desirable to create a surface, with higher number of reactive sites, that serves as a matrix on a solid support for the attachment of protein capture agents.

This has been achieved by the attachment of specific polymers to the support, which serve as “scaffolds” for the attachment of biological probes. This approach is reported in U.S. Ser. No. 10/091,644 (Docket 83598), U.S. Pat. No. 5,858,653 and EP 1 106 603. The manufacture of these supports, however, is laborious and costly.

Another approach involves the use of a support with enhanced surface area. U.S. patent application No. 2002142339, WO2001016376 report the use of a porous glass support for this purpose. Similarly, Nylon porous membranes on glass have been disclosed in U.S. patent application Ser. No. 2002119559 and WO 2002002585, as well as porous membranes made by a phase separation process in U.S. patent application No. 2002086307. WO 00/61282 reports biological microarray supports comprised of porous silica, which may be prepared by both additive and subtractive processes.

There remains a need for a diagnostic method, which provides a support for biological microarrays, which in turn allows for the attachment of high levels of biological affinity agents.

### PROBLEM TO BE SOLVED

The problem to be solved is to improve the immobilization capacity of biological microarrays in a manner that lends itself to low cost—high volume coating manufacturing methods.

### SUMMARY OF THE INVENTION

The present invention relates to a microarray comprising a support having attached to a surface thereof at least one porous layer, wherein the porous layer comprises a hydrophilic binder and polymer particles. The present invention also relates to a method of using a microarray comprising providing a microarray comprising a support having attached to a surface thereof at least one porous layer, wherein the porous layer comprises a hydrophilic binder and polymer particles; contacting the microarray with biological targets labeled with optical emission tag; and measuring the signals from the optical emission tag.

### ADVANTAGEOUS EFFECT OF THE INVENTION

The present invention includes several advantages, not all of which are incorporated in a single embodiment. The biological microarrays of this invention allow for high loading levels of biological affinity tags. The microarrays also may be prepared using coating methods, which may easily be scaled to high volume production. Also, in some embodiments of this invention, no coupling reagents are needed for the attachment of biological molecules to the microarray.

### BRIEF DESCRIPTION OF THE DRAWINGS

FIG. 1 illustrates particles P-4, analyzed by electron microscopy, which appear as highly deformed spherical particles with deep ridges and wrinkles.

FIG. 2 illustrates particles P-5, analyzed by electron microscopy, which appear as spherical particles with wrinkled surfaces.

### DETAILED DESCRIPTION OF THE INVENTION

The present invention relates to a microarray comprising a support having attached to a surface thereof at least one porous layer. The porous layer comprises more than a single monolayer of polymer particles and a hydrophilic binder. The present invention also relates to a method of using a microarray comprising providing a microarray comprising a support having attached to a surface thereof at least one porous layer, contacting the microarray to biological targets labeled with optical emission tag, and measuring optical emission tag signals.

The porous layer contains a continuous network of interstitial voids between the particles. The layer is of a thickness such that, on average, more than one monolayer and, preferably, at least five monolayers of particles are packed one atop another in the vertical dimension. In a preferred embodiment, chemically active groups capable of binding bioaffinity tags are present either on the binder, on the particles, or both.

In general, a biological microarray may be prepared by first modifying a support, preferably the protein microarray support, followed by depositing various biological capture agents onto the modified support at pre-defined locations. Although the preferred embodiment described herein is referred to as a protein microarray, the present invention may also be applied to nucleic acid microarrays. As used herein, the term “microarray” means a 2 dimensional pattern consisting of a plurality of biological capture agents immobilized in a spatially addressable manner. Although a typical microarray pattern is 2 dimensional, the pattern used in the present invention is 3-dimensional, that is, more than a single monolayer of polymer particles in thickness. The term “support” means a material having a rigid or semi rigid surface and at least one side of the support surface is substantially flat. The terms “biological capture agent”, “biological affinity tag” and “bioaffinity tag” mean a molecule that can interact with biological target compounds in high affinity and high specificity. One variety of biological capture agent is referred to herein as a protein capture agent. The term “protein capture agent” means a molecule that can interact with proteins in high affinity and high specificity. Typically it is desirable to have an affinity binding constant between a protein capture agent and target protein greater than 10⁶ M⁻¹.

The supports for use in the present invention may be transparent or opaque, flexible or rigid. Glass, or fused silica, is the most commonly used microarray support in the art, although plastics, metals, and semiconductors may also be used. Generally, a glass support is planar and has high flatness and clarity. Preferably, the glass does not fluoresce and has a thickness from 0.1 mm to 5 mm. The glass support may have any dimensions and may be cut into various sizes, according to its intended uses. The support used in the invention may also be any of those usually used in the art, such as resin-coated paper, paper, polyesters, or microporous materials such as polyethylene polymer-containing material sold by PPG Industries, Inc., Pittsburgh, Pa. under the trade name of Teslin®, Tyvek® synthetic paper (DuPont Corp.), and OPPalyte® films (Mobil Chemical Co.) and other composite films listed in U.S. Pat. No. 5,244,861. Opaque supports include plain paper, coated paper, synthetic paper, photographic paper support, melt-extrusion-coated paper, and laminated paper, such as biaxially oriented support laminates. Biaxially oriented support laminates are described in U.S. Pat. Nos. 5,853,965; 5,866,282; 5,874,205; 5,888,643; 5,888,681; 5,888,683; and 5,888,714, the disclosures of which are hereby incorporated by reference.

These biaxially oriented supports include a paper base and a biaxially oriented polyolefin sheet, typically polypropylene, laminated to one or both sides of the paper base. Transparent supports include glass, cellulose derivatives, such as a cellulose ester, cellulose triacetate, cellulose diacetate, cellulose acetate propionate, cellulose acetate butyrate, polyesters, such as poly(ethylene terephthalate), poly(ethylene naphthalate), poly-1,4-cyclohexanedi-methylene terephthalate, poly(butylene terephthalate), and copolymers thereof, polyimides, polyamides, polycarbonates, polystyrene, polyolefins, such as polyethylene or polypropylene, polysulfones, polyacrylates, polyether imides, and mixtures thereof. The term as used herein, “transparent” means the ability to pass radiation without significant deviation or absorption. Although the support of choice for protein microarray applications may be organic, inorganic or biological, glass is preferred. In some cases, the support may be a porous membrane, for example, nitrocellulose and polyvinylidene difluoride, and protein capture agents may be deposited onto the membrane by physical adsorption.

The polymer particles used in preparing the present invention comprise one or more polymers each of which is prepared from one or more ethylenically unsaturated polyrnerizable monomers. In some embodiments, the particles may be homogeneous, that is, they are composed of the same polymer throughout. In other embodiments, the particles may be composed of two or more polymers or monomers, for example, as core/shell particles, as described, for example, in U.S. Pat. No. 4,401,765. The particles used in the present invention may contain chemically active groups, most preferably vinylsulfonyl units, which may be present on the surface of the particle or on soluble polymer stabilizer molecules extending from the particle surface.

These particles may have a mean diameter of from 0.05 to 50 microns. Preferably, the mean diameter is from 0.25 to 10 microns. Most preferably, the mean diameter is from 0.50 to 5 microns. Preferably these particles will be monodisperse or relatively monodisperse. “Monodisperse” means that the coefficient of variation of the particle size distribution, that is, the standard deviation as a percentage of the mean, will be less than 20%. More preferably, the coefficient of variation will be less than 15%. Most preferably, the coefficient of variation will be less than 10%.

The particular polymer, which comprises the particles, may be a water insoluble synthetic polymer. This polymer may be of any class, provided that it is water-insoluble and may be prepared as a particulate dispersible in a useful carrier solvent through any known procedure. Such classes include, but are not limited to addition polymers, poly (alkylene oxides), phenol-formaldehyde polymers, urea-formaldehyde polymers and condensation polymers consisting of one or more of the following repetitive units: esters, amides, imides, carbonates, urethanes, and ethers.

Preferably this polymer will be an addition polymer of monomers containing α,β-ethylenic unsaturation. These include, but are not necessarily limited to methacrylic acid esters, such as methyl methacrylate, ethyl methacrylate, isobutyl methacrylate, 2-ethylhexyl methacrylate, benzyl methacrylate, phenoxyethyl methacrylate, cyclohexyl methacrylate and glycidyl methacrylate, acrylate esters such as methyl acrylate, ethyl acrylate, isobutyl acrylate, 2-ethylhexyl acrylate, benzyl methacrylate, phenoxyethyl acrylate, cyclohexyl acrylate, and glycidyl acrylate, styrenics such as styrene, ax-methylstyrene, 3- and 4-chloromethylstyrene, halogen-substituted styrenes, and alkyl-substituted styrenes, vinyl halides and vinylidene halides, N-alkylated acrylamides and methacrylamides, vinyl esters such as vinyl acetate and vinyl benzoate, vinyl ether, allyl alcohol and its ethers and esters, and unsaturated ketones and aldehydes such as acrolein and methyl vinyl ketone, isoprene, butadiene and acrylonitrile. Preferably, the monomers will be styrenics or acrylic esters or methacrylic esters.

In addition, small amounts, typically less than 20% of the total weight of the polymerizeable solids, of one or more water-soluble ethylenically unsaturated monomers may be used. Such monomers may include styrenics, acrylates, and methacrylates substituted with highly polar groups, unsaturated carbon and heteroatom acids such as acrylic acid, methacrylic acid, fumaric acid, maleic acid, itaconic acid, vinylsulfonic acid, vinylphosphonic acid, and their salts, vinylcarbazole, vinylimidazole, vinylpyrrolidone, and vinylpyridines.

The polymer particles of this invention may further comprise monomers containing at least two ethylenically unsaturated chemical functionalities. These functionalities may be vinyl groups, acrylates, methacrylates, vinyl ethers and vinyl esters. Monomers include, but are not limited to trimethylolpropane triacrylate, ethylene glycol dimethacrylate, isomers of divinylbenzene, and ethylene glycol divinyl ether.

In a preferred embodiment, the polymer particles are rich in specific functionalities, which impart dispersibility in a desired carrier solvent, compatibility with the microarray's matrix, or the ability to form chemical bonds with biological capture agents. These chemical functionalities may be present on the surface of the particle or on stabilizer polymer strands, which are covalently grafted, chemisorbed, or physically adsorbed to the particle surface. These chemical functionalities may be, but are not limited to thiols, primary amines, secondary amines, tertiary amines, quaternary ammoniums, phosphines, alcohols, carboxylic acids, vinylsuflonyls, aldehydes, epoxies, hydrazides, succinimidyl esters, carbodiimides, maleimides, iodoacetyls, isocyanates, isothiocyanates, aziridines, sulfonates. Preferably, these functionalities will be carboxylic acids, primary amines, secondary amines, or carboxylic acids.

In a preferred embodiment, these reactive functional units will be present on soluble stabilizer polymers, which are covalently grafted, chemisorbed, or adsorbed to the surface of the polymer particles. Stabilizer polymers, which may be used for this purpose, include addition polymers and copolymers. Especially preferred stabilizer polymers may include poly (propyleneimine) and polymers and copolymers of methacrylic acid, acrylic acid, mercaptomethyl styrene, N-aminopropyl (meth)acrylamide and secondary amine derivatives thereof, N-aminoethyl (meth)acrylate and secondary amine forms thereof, diallyamine, vinylbenzylamine, vinylamine, (meth)acrylic acid, vinylbenzyl mercaptan, and hydroxyethyl(meth)acrylate. Preferably, the polymer is poly(vinylamine), poly(propyleneimine), polyethyleneimine, polyacrylic acid, polymethacrylic acid, or poly(N-aminopropyl methacrylamide).

In an especially preferred embodiment, the polymer particles contain stabilizer polymer comprising pendant vinylsulfonyl or latent vinylsulfonyl groups. Stabilizer polymers having activated vinylsulfonyl groups possess additional advantages in that proteins may be attached to the polymers under milder conditions and utilize less process control during manufacture. This renders manufacture more efficient and less costly. Formula I represents a generalized structure for a stabilizer polymer containing vinylsulfonyl groups or vinylsulfonyl precursor polymers.

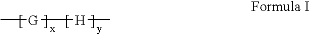

Polymers represented by the structure in Formula I, consist of the polymerization products of a “G” monomer, which affords favorable solubility and/or physical properties to the polymer, and an “H” monomer, which contains the vinylsulfone moiety or, more preferably, a vinylsulfone precursor function, such as a sulfonylethyl group with a leaving group in the β-position. x and y both represent molar percentages ranging from 10 to 90 and 90 to 10. Preferably, x and y range from 25 to 75 and 75 to 25, respectively.

G is a polymerized α,β-ethylenically unsaturated addition polymerizeable monomer which imparts desirable solubility properties to the polymer or which allows the polymer particles of this invention to be readily dispersed in a carrier solvent (water in most cases) or readily grafted or immobilized within a matrix or on a solid support. The monomer from which polymerized G may be derived include both ionic and nonionic monomers. Ionic monomers may include, for example, anionic ethylenically unsaturated monomers such as 2-phosphatoethyl acrylate potassium salt, 3-phosphatopropyl methacrylate ammonium salt, acrylamide, methacrylamides, maleic acid and salts thereof, sulfopropyl acrylate and methacrylate, acrylic and methacrylic acids and salts thereof, N-vinylpyrrolidone, acrylic and methacrylic esters of alkylphosphonates, styrenics, acrylic and methacrylic monomers containing amine ammonium functionalities, styrenesulfonic acid and salts thereof, acrylic and methacrylic esters of alkylsulfonates, vinylsulfonic acid and salts thereof. Nonionic monomers may include monomers containing hydrophilic, nonionic units such as poly(ethylene oxide) segments, carbohydrates, amines, amides, alcohols, polyols, nitrogen-containing heterocycles, and oligopeptides. Examples include, but are not limited to poly(ethylene oxide) acrylate and methacrylate esters, vinylpyridines, hydroxyethyl acrylate, glycerol acrylate and methacrylate esters, (meth)acrylamide, and N-vinylpyrrolidone.

Preferably, G is the polymerized form of acrylamide, sodium 2-acrylamido-2-methanepropionate, sulfopropyl acrylate and methacrylate salts, or sodium styrenesulfonate.

Monomer H is preferably a vinylsulfone or vinylsulfone precursor unit covalently bound to a polymerizeable α,β-ethylenically unsaturated function by an organic spacer, which consists of Q and L, of which Q is an optional component, as represented by Formula II. 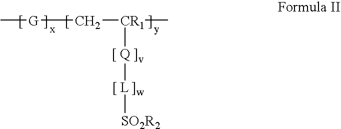
Wherein:
R₁ is a hydrogen atom or a C₁-C₆ alkyl group. Preferably R₁ is a hydrogen atom.
Q is —CO₂—, or CONR₁.;
v is 1 or 0;
w is 1-3;
L is a divalent linking group containing at least one linkage selected from the group consisting of —CO₂— and —CONR₁, and containing 3-15 carbon atoms, or a divalent atom containing at least one linkage selected from the group consisting of —O—, —N(R₁)—, —CO—, —SO—, —SO₂—, —SP₃—, —SO₂N(R₁)—, —N(R₁)CON(R₁)— and —N(R₁)CO₂—, and containing 1-12 carbon atoms in which R₁ has the same meaning as defined above;
R₂ is —CH═CH₂ or —CH2-CH2X₁ wherein X₁ is a substituent replaceable by a nucleophilic group or releasable in the form of HX₁ by a base. X₁ may be, but is not necessarily limited to —S₂O₃—, —SO₄—, —Cl, —Br, —I, quaternary ammonium, pyridinium, —CN, and sulfonate esters (such as mesylate and tosylate).

Vinylsulfone and vinylsulfone-containing precursor “H” monomers useful in this embodiment include, but are not necessarily limited to those compounds disclosed in U.S. Pat. Nos. 4,548,869 and 4,161,407 (incorporated herein by reference). Preferred vinylsulfone and vinylsulfone-containing precursor “H” monomers useful in this embodiment include the following structures:

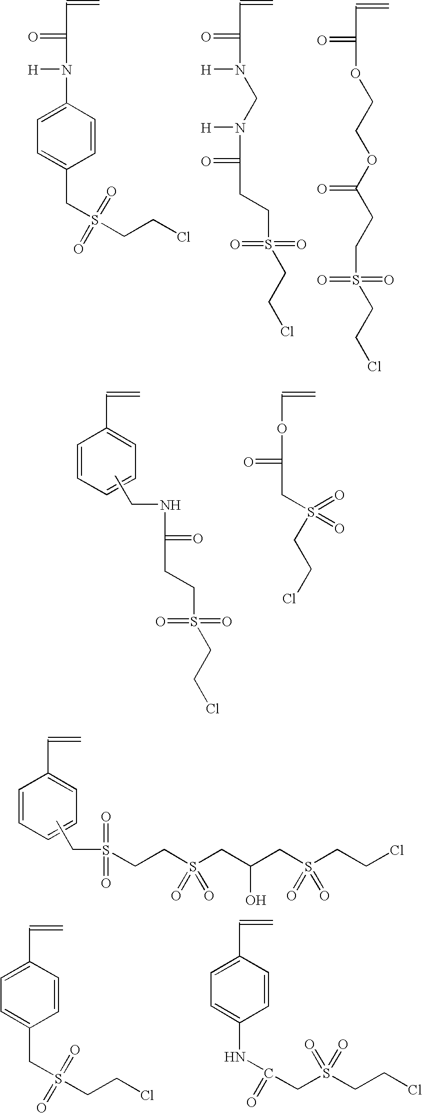

More than one type each of G and H monomers may be present in the same polymer. Additional monomers may be incorporated in order to modify properties such as glass transition temperature, surface properties, and compatibility with other formulation components as needed for specific applications. Selection of additional monomers will be application dependent and will be obvious to one skilled in the art.

As noted above, the polymer particles useful in the practice of this invention may be homogeneously composed of one of the polymers, or a mixture thereof. Alternatively, the polymers may be an outer graft or shell of a grafted copolymer or core-shell particle, respectively. Useful core-shell polymers are described, for example, in U.S. Pat. No. 4,997,772.

The polymeric particles may be prepared using any suitable heterogeneous polymerization technique. Such techniques are reviewed in Arshady, R. “Suspension, Emulsion, and Dispersion Polymerization: a Methodological Survey” Coloid. Polym. Sci. 1992, 270, 717-732 and in Lovell, P. A.; El-Aaser, M. S. “Emulsion Polymerization and Emulsion Polymers”,; Wiley: Chichester, 1997. These techniques include emulsion techniques, such as batch, semi-continuous, and continuous techniques, and suspension polymerization techniques, limited coalescence suspension techniques, dispersion polymerization, miniemulsion polymerization and other techniques known to those skilled in the polymer chemistry art. Surfactantless emulsion polymerization is preferred, as it may be used to provide highly monodisperse particles without the use of surfactants or emulsifiers as described, for example, in Wang, P. H.; Pan, C. Y. “Preparation of Styrene/Acrylic Acid copolymer Microspheres: Polymerization Mechanism and Carboxyl Group Distribution” Colloid Polym. Sci. 2002, 280, 152-159, and Zeng, F.; Sun, F.; Wu, S.; Liu, X.; Wang, Z.; Tong, Z. “Preparation of Highly Charged Monodisperse nanospheres.” Macromol. Chem. Phys. 2002, 203, 673-677. Dispersion polymerization is an especially preferred technique, as it may afford highly monodisperse polymeric particles with reactive stabilizer polymers grafted to the surface.

Staged emulsion polymerization may be used to provide a core-shell polymer composed of two different polymers. Emulsion polymerization of the core is carried to substantial completion by continuously adding reactants to a reaction vessel under standard conditions. Monomers and catalysts needed to make the shell polymer are then continuously added to the vessel containing the latex of the core polymer. In this manner, the shell has a definite known composition rather than being a mixture of core and shell monomers. Representative details of preparing the core-shell polymeric particles useful in this invention are provided in U.S. Pat. No. 4,997,772.

In addition, polymer particles may be prepared via solvent evaporation methods. In these methods, a pre-formed, water-insoluble polymer is dissolved in a water-miscible or water-immiscible solvent and combined with water in the presence of a stabilizing species such as a surfactant, an emulsifier, or a naturally occurring or synthetic polymer, resin, or gum with amphiphilic character. The mixture is emulsified using a high shear mixing technique and the solvent is evaporatively removed to afford polymer particles dispersed in water.

The polymer particles are bound to each other and to the surface of the support by a hydrophilic binder. Examples include materials such as gelatin, water-soluble cellulose ethers, poly(n-isopropylacrylamide), polyvinylpyrrolidone and vinylpyrrolidone-containing copolymers, polyethyloxazoline and oxazoline-containing copolymers, imidazole-containing polymers, polyacrylamides and acrylamide-containing copolymers, poly(vinyl alcohol) and vinyl-alcohol-containing copolymers, poly(vinyl methyl ether), poly(vinyl ethyl ether), poly(ethylene oxide), acacia, alginic acid, bentonite, carbomer, carboxymethylcellulose sodium, cetostearyl alcohol, colloidal silicon dioxide, ethylcellulose, guar gum, hydroxyethylcellulose, hydroxypropyl cellulose, hydroxypropyl methylcellulose, magnesium aluminum silicate, maltodextrin, methylcellulose, povidone, propylene glycol alginate, sodium alginate, sodium starch glycolate, starch, tragacanth, xanthum gum, and mixtures thereof. Further discussion on hydrophilic binders, which may include gelling agents, may be found in Secundum Artem, Vol. 4, No. 5, Lloyd V. Allen. A preferred binder is alkali-pretreated gelatin.

In a preferred embodiment, the hydrophilic polymer binder is rich in specific functionalities such as, for example, chemically active groups. In a preferred embodiment, the hydrophilic polymer binder will be made from a precursor polymer rich in such reactive units as thiols, primary amines, secondary amines, tertiary amines, phosphines, alcohols, carboxylic acids, vinylsulfonyls, aldehydes, epoxies, hydrazides, succinimidyl esters, carbodiimides, maleimides, iodoacetyls, isocyanates, isothiocyanates, or aziridines. Preferably the reactive unit is a primary or secondary amine or a vinylsulfonyl. Specific polymers which may be used for this purpose may be selected from the set consisting of, but not necessarily limited to, poly (propyleneimine) and polymers and copolymers of N-aminopropyl (meth)acrylamide and secondary amine derivatives thereof, N-aminoethyl (meth)acrylate and secondary amine forms thereof, diallyamine, vinylbenzylamine, vinylamine, (meth)acrylic acid, vinylbenzyl mercaptan, and hydroxyethyl(meth)acrylate. Preferably, the polymer is poly(vinylamine), poly(propyleneimine), or poly(N-aminopropyl methacrylamide).

The porous layer may also include crosslinking agents. Any crosslinking agent may be used provided its reactive functionalities have the appropriate reactivity with specific chemical units in the binder. Some common crosslinkers which may crosslink binders rich in lewis basic functionalities include, but are not necessarily limited to, carbodiimides, polyvalent metal cations, organic isocyanates such as tetramethylene diisocyanate, hexamethylene diisocyanate, diisocyanato dimethylcyclohexane, dicyclohexylmethane diisocyanate, isophorone diisocyanate, dimethylbenzene diisocyanate, methylcyclohexylene diisocyanate, lysine diisocyanate, tolylene diisocyanate, diphenylmethane diisocyanate, aziridines such as taught in U.S. Pat. No. 4,225,665, ethyleneimines such as Xama-7® sold by EIT Industries, blocked isocyanates such as CA BI-12 sold by Cytec Industries, melamines such as methoxymethylmelamine as taught in U.S. Pat. No. 5,198,499, alkoxysilane coupling agents including those with epoxy, amine, hydroxyl, isocyanate, or vinyl functionality, Cymel® crosslinking agents such as Cymel 300®, Cymel 303®, Cymel 1170®, Cymel 1171® sold by Cytec Industries, and bis-epoxides such as the Epong family sold by Shell. Other crosslinking agents include compounds such as aryloylureas, aldehydes, dialdehydes and blocked dialdehydes, chlorotriazines, carbamoyl pyridiniums, pyridinium ethers, formamidinium ethers, vinyl sulfones, boric acid, dihydroxydioxane, and polyfunctional aziridines such as CX-100 (manufactured by Zeneca Resins). Such crosslinking agents maybe low molecular weight compounds or polymers, as discussed in U.S. Pat. No. 4,161,407 and references cited therein.

The coating mixture of polymer particles, binder, and other optional addenda may be coated by conventional coating means onto a support material commonly used in this art. Coating methods may include, but are not limited to, wound wire rod coating, knife coating, slot coating, slide hopper coating, gravure coating, spin coating, dip coating, skim-pan-air-knife coating, multilayer slide bead, doctor blade coating, gravure coating, reverse-roll coating, curtain coating, or multilayer curtain coating. Some of these methods allow for simultaneous coatings of more than one layer, which is preferred from a manufacturing economic perspective if more than one layer or type of layer needs to be applied. Known coating and drying methods are described in further detail in Research Disclosure no. 308119, published December 1989, pages 1007 to 1008. Coating methods are also broadly described by Edward Cohen and Edgar B. Gutoff in Chapter 1 of “Modern Coating And Drying Technology”, (Interfacial Engineering Series; v.1), (1992), VCH Publishers Inc., New York, N.Y. For a single layer format, suitable coating methods may include dip coating, rod coating, knife coating, blade coating, air knife coating, gravure coating, forward and reverse roll coating, and slot and extrusion coating. The support may be stationary, or may be moving so that the coated layer or layers is immediately drawn into drying chambers. After coating, the layers are generally dried by simple evaporation, which may be accelerated by known techniques such as convection or microwave heating. Generally, the coated layer thickness for the porour layer will be from 0.25 to 250 microns, preferably from 1.25 to 50 microns, and most preferably from 2.5 to 25 microns.

In order to obtain adequate coatability, additives known to those familiar with such art such as surfactants, defoamers, and alcohol may be used. Coating aids and surfactants include, but are not limited to, nonionic fluorinated alkyl esters such as FC-430®, FC-431®, FC-10®, FC-171®V sold by Minnesota Mining and Manufacturing Co., Zonyl® fluorochemicals such as Zonyl-FSN®, Zonyl-FTS®, Zonyl-TBS®, Zonyl-BA® sold by DuPont Corp., other fluorinated polymer or copolymers such as Modiper F600® sold by NOF Corporation, polysiloxanes such as Dow Corning DC 1248®, DC200®, DC510®, DC 190®, BYK 320®, BYK 322®, sold by BYK Chemie, SF 1079®, SF1023®, SF 1054®, and SF 1080® sold by General Electric, the Silwet® polymers sold by Union Carbide, polyoxyethylene-lauryl ether surfactants, sorbitan laurate, palmitate and stearates such as Spang surfactants sold by Aldrich, poly(oxyethylene-co-oxypropylene) surfactants such as the Pluronic® family sold by BASF, other polyoxyethylene-containing surfactants such as the Triton X® family sold by Union Carbide, ionic surfactants, such as the Alkanol® series sold by DuPont Corp., and the Dowfax® family sold by Dow Chemical. Specific examples are described in MCCUTCHEON's Volume 1: Emulsifiers and Detergents, 1995, North American Edition.

In order to improve the adhesion of the layer to the support, an under-coating or subbing layer may be applied to the surface of the support. This layer may be an adhesive layer such as, for example, halogenated phenols, partially hydrolyzed vinyl chloride-co-vinyl acetate polymer, vinylidene chloride-methyl acrylate-itaconic acid terpolymer, a vinylidene chloride-acrylonitrile-acrylic acid terpolymer, or a glycidyl (meth)acrylate polymer or copolymer. To immobilize protein capture agents, the support may be coated with an adhesive interlayer and further coated with an upper layer wherein certain chemical functional groups are incorporated. Other chemical adhesives, such as polymers, copolymers, reactive polymers or copolymers, that exhibit good bonding between the porous layer and the support may be used.

The polymeric binder in a subbing layer, which may be employed in the invention, is preferably a water soluble or water dispersible polymer such as poly(vinyl alcohol), poly(vinyl pyrrolidorie), gelatin, a cellulose ether, a poly(oxazoline), a poly(vinylacetamide), partially hydrolyzed poly(vinyl acetate/vinyl alcohol), poly(acrylic acid), poly(acrylamide), poly(alkylene oxide), a sulfonated or phosphated polyester or polystyrene, casein, zein, albumin, chitin, chitosan, dextran, pectin, a collagen derivative, collodian, agar-agar, arrowroot, guar, carrageenan, tragacanth, xanthan, rhamsan; a latex such as poly(styrene-co-butadiene), a polyurethane latex, a polyester latex, or a poly(acrylate), poly(methacrylate), poly(acrylamide) or copolymers thereof. Other methods to improve the adhesion of the layer to the support include surface treatment of the support by corona discharge plasma-treatment in a variety of atmospheres and UV treatment, which is performed prior to applying the layer to the support.

Once a support is modified by the porous layer, the biological capture agents, preferably protein capture agents, will be deposited onto the support in a spatially addressable manner to generate biological microarray content. The biological capture agent may be bound to the hydrophilic binder of the porous layer, the polymer particle of the porous layer, the stabilizer polymer or a combination thereof.

A protein molecule consists of 20 amino acids that are connected in linear manner covalently. Some proteins may be further modified at selected amino acids through posttranslational processes that include phosphorylation and glycosylation. A protein molecule may be used as a protein capture agent. There are several classes of molecules that may be used as protein capture agents on a protein microarray. Antibodies are a class of naturally occurring protein molecules that are capable of binding targets with high affinity and specificity. The properties and protocols of using antibody can be found in “Using Antibodies; A Laboratory Manual”, (Cold Spring Harbor Laboratory Press, by Ed Harlow and David Lane, Cold Spring Harbor, N.Y. 1999). Antigens may also be used as protein capture agents if antibodies are intended targets for detection. Protein scaffolds, such as whole protein/enzyme or their fragments, may be used as protein capture agents as well. Examples include phosphotases, kinases, proteases, oxidases, hydrolyases, cytokines, or synthetic peptides. Nucleic acid ligands may be used as protein capture agent molecules after in vitro selection and enrichment for their binding affinity and specificity to certain targets. The principle of such selection process can be found in Science, Vol. 249, 505-510, 1990 and Nature, Vol. 346, 818-822, 1990. U.S. Pat. No. 5,110,833 discloses an alternative class of synthetic molecules that may mimic antibody binding affinity and specificity and may be readily prepared by the Molecular Imprinting Polymer (MIP). This technology has been reviewed in Chem. Rev. Vol. 100, 2495-2504, (2000). Preferably, the biological capture agent, or bioaffinity tag, comprises DNA, antibodies, antigens, proteins, enzymes, nucleic ligands, and polysaccharides.

Normally the protein capture agents are dissolved in aqueous solution or a buffer to the desirable concentrations. The protein capture agents may be deposited on a microarray support in a spatially addressable manner using commercially available robotic spotting machine. The volume of a protein capture agent solution per spot may vary anywhere from picoliter to nanoliter, depending on the choice of spotting methods. Some commonly used spotting methods may include pin spotting, quill spotting, inkjet spotting. The spotting may be either contact spotting or non-contact spotting and may be performed either manually or robotically.

The invention further discloses a process of using such microarray. In a typical microarray analysis process, a biological sample solution containing a mixture of targets is non-selectively labeled using “emission tags”, wherein “target” refers to a molecule, typically a macromolecule, such as a polypeptide, or polysaccharides, whose presence, amount, and/or identity are to be determined. Some commonly used emission tags include, but are not limited to, fluorescers, chemiluminescers, radioactive molecules, enzymes, enzyme supports, and other spectroscopically detectable labels. Alternatively, a molecule that can emit fluorescence, chemiluminescence, or spectroscopicily detectable signals upon binding with other molecules may also be used as emission tags. Once an emission tag has been selected, the methods of labeling nucleic acids have been described in BioTechnology 6:816-821, (1988) by Sambrook et al, and in Nuc. Acids Res. 13:2399-2412, (1985) by Smith, L. et al; the methods of labeling polypeptides have been described in chapter 5 of Sequencing of Proteins and Peptides, by Allen, G., Elsevier, New York (1989) and in Chemistry of the Amino Acids, by Greenstein and Winitz, Wiley and Sons, New York (1961); and the methods of labeling polysaccharides have been described in Carbohydrate Analysis: A practical Approach, by Chaplin and Kennedy, IRL Press, Oxford (1986). After the target analytes in a biological sample are labeled with emission tags, they may be hybridized to the polymer particle based microarray.

In practice, a protein microarray is brought into contact with a mixture of emission tag labeled protein targets, protein targets in the sample will adsorb to both areas spotted with specific protein capture agents and areas without protein capture agents. Since the protein microarray is intended to be used for the measurement of specific interactions between protein capture agents on the microarray with certain proteins or other molecules in the biological fluid sample, the non-specific binding of sample proteins to non-spotted area would give rise to high background noise. The term non-specific binding refers to the tendency of protein molecules to adhere to a solid surface in a non-selective manner. This high background noise resulting from the non-specific binding will interfere with reporter signals to be detected from the spotted area unless the non-specific binding is blocked in an appropriate manner.

Typically, the protein microarray will be immersed in a solution containing a blocking agent to block the non-specific binding sites before its contact with the intended analyte solution. A commonly used method for blocking protein non-specific binding is to treat the surface of the support with a large excess of bovine serum albumin. The non-spotted surface area may also be chemically modified with polyethylene glycol (PEG), phospholipid, or poly lysine to prevent non-specific binding. After blocking, the unbound protein targets may be washed away using a buffer solution.

The emission tag signals resulting from the interaction between labeled analytes and the protein capture agents on the surface of the microarray may be measured using a scanner or other imaging instrument. An alternative method of detecting the specific binding between a protein capture agent and a protein target includes labeling of the protein capture agent with an emission tag. In this way, the protein capture agents immobilized on the support will interact with unlabeled target analytes first to form complexes in spots, followed by immersing the microarray in a solution containing emission tag labeled protein capture agents, or a mixture of emission tag labeled protein capture agents. Thus, a sandwich complex is formed, in which the complex contains a protein capture agent bound to a target and the target further bound to another emission tag labeled protein capture agent. When an antibody is used as protein capture agent and an enzyme is used as emission tag, the method has been described in detailed in “Using Antibodies; A Laboratory Manual”, (Cold Spring Harbor Laboratory Press, by Ed Harlow and David Lane, Cold Spring Harbor, N.Y. 1999). The emission tag signals resulting from the formation of the sandwich complex on the surface of the microarray may be measured using a scanner or other imaging instrument.

### EXAMPLES

The invention may be better appreciated by reference to the following specific embodiments.

### Preparation of Polymer Particle P-1: Polystyrene Particles Stabilized by vinylsulfone-Containing Polymers Grafted to the Surface made via a Three Step Synthesis

### Step 1: Synthesis of chloroethylsulfone-Containing Stabilizer Precursor Polymer

N-[4-[[(2-chloroethyl)sulfone]methyl]phenyl]acrylamide (22.5 g), sodium 2-acrylamido-2-methanepropionate (34.5 g of a 52.2% w/w solution in water), and 4,4′-azobis(cyanovaleric acid) (0.76 g) were dissolved in 95.0 g N-methyl pyrrolidinone in a 500 mL 3-neck round bottom flask outfitted with a mechanical stirrer, condenser, and nitrogen inlet. The solution was bubble degassed with nitrogen for 10 minutes and heated for 16 hours at 65° C. The resulting solution was precipitated into 3 L propyl acetate to produce a white, sticky semisolid from which the solvents were decanted. The crude product was redissolved in 300 ml methanol and precipitated again into 3 L isopropyl ether. The resulting tacky solid was isolated by decanting the solvents and was dried in a vacuum oven at 80° C. for 48 hours to afford 42.5 g of a white powder. The chloroethylsulfone content of the polymer was determined to be 1.732 mEq/g by titration with NaOH, which is equivalent to a polymer 44.3 mol% of N-[4-[[(2-chloroethyl)sulfone]methyl]phenyl]acrylamide monomer. Size exclusion chromatography (SEC) of the polymer in hexafluoroisopropanol gave absolute molecular weights of Mn=33,800 and Mw=96,300.

### Step 2:Preparation of Polystyrene Particles Stabilized by chloroethylsulfone-Containing Precursor Polymer of Step 1

The chloroethylsulfone-containing polymer of Step 1 (3.50 g) was dissolved in methanol (200.00 g) in a 500 ml three neck round bottom flask with a reflux condenser, nitrogen inlet, and mechanical stirrer. The solution was bubble degassed with nitrogen for 20 minutes and the reaction vessel was placed in a thermostatted water bath at 52° C.. A similarly degassed solution of 2,2′-azobis(2,4-dimethylvaleronitrile) (0.20 g) in styrene (25.00 g, passed over basic alumina) was added all at once and the reaction was allowed to stir at 250 RPM overnight (about 16 hours). After about 20 minutes, the reaction became a translucent blue. The crude, white product latex was purified by three cycles of centrifugation, decantation of the clear supernatant, and redispersion in methanol. The final redispersion step used deionized water. 78.67 g of a 28.63% solids dispersion was obtained. The mean particle size was determined by the Coulter counter method (mean=1.40 μm. CV=23.56%). The loading of chloroethylsulfonyl functionalities was determined by a titrimetric procedure. An aliquot of the bead dispersion was added to a known quantity of aqueous NaOH, allowed to react for 30 minutes at room temperature, and back titrated with HCl (4.2×10⁻³ meq vinylsulfone/g solid beads).

### Step 3: Elimination of chloroethylsulfone Units

To 54.79 g of the particle dispersion of Step 2 was added 0.21 g 1.00 N NaOH. After 30 minutes, the pH had stabilized at 7.

### Preparation of Polymer Particle P-2: Synthesis of poly(styrene-co-methacrylic acid) Particles Via Surfactantless Latex Polymerization

Sodium chloride (0.65 g) and methacrylic acid (1.70 g) were dissolved in triple filtered water (700 mL) in a 3-neck 1 L round bottom flask equipped with a mechanical stirrer, reflux condenser, and nitrogen inlet. Styrene (91.6 mL, passed over basic alumina) was added and the mixture was bubble degassed with nitrogen for 30 minutes at room temperature. The reaction vessel was immersed in a thermostatted water bath at 75° C. and was bubble degassed for 15 more minutes. A bubble degassed solution of potassium persulfate (0.50 g) in triple filtered water (65.0 mL) was added all at once and stirring was initiated at 350 RPM. After about 5 minutes, a slight bluish tinge was evident in the reaction. The reaction was allowed to proceed for 24 hours and was then filtered through cheesecloth. The latex was purified by ultrafiltration through a 100K cutoff membrane with four volumes of water. 749.00 g of a latex of 9.9% solids was obtained. The dispersion was roto-evaporated until it reached a concentration of 21.68 wt. % solids. The mean particle diameter was determined using a Horiba LA-920 particle analyzer to be 0.428 μm with a coefficient of variation of 15.88%.

### Preparation of Polymer Particle P-3: Synthesis of polyester-Containing Polymer Particles with Quaternary Ammonium Moieties

Fineclad® 385 unsaturated polyester resin (90.0 g, available from Reichhold Inc.) was dissolved in a solution of divinylbenzene (45.0 g, 80% mixture of isomers with remainder being ethylstyrene, passed over basic alumina), chloromethylstyrene (45.0 g, passed over basic alumina), toluene (180.0 g) and hexadecane (7.2 g) at 40° C. and the solution was cooled to room temperature. Azobis(isobutyronitrile) (1.8 g) was then added and stirred until dissolved. An aqueous phase was prepared consisting of a dodecanethiol-endcapped acrylamide decamer (14.4 g, prepared by the procedure described in U.S. Pat. No. 6,127,453 (column 9, lines 40-55)) dissolved in 1080 gm. deionized water. The two phases were combined and emulsified, first using a Silverson L4R mixer on the highest power setting for 1-2 minutes, then by passage twice through a M-110T Microfluidizer (sold by Microfluidics). The resulting microsuspension was transferred to a 3-neck round bottom flask equipped with a condenser, mechanical stirrer, and nitrogen inlet and was bubble degassed with nitrogen for 10 minutes. The reaction was then stirred for 16 hours in a thermostatted water bath at 70° C.. The toluene was removed via rotary evaporation and N,N-dimethylethanolamine (26.3 g) was added. The reaction was then heated overnight at 70° C., dialyzed using a 14K membrane, and freeze dried to yield powder (142.5 g). The powder was redispersed in water using sonication for two minutes to give a final dispersion of 20 wt. % solids. The mean particle diameter was determined using a Horiba LA-920 particle analyzer to be 0.300 gm with a coefficient of variation of 16.07%.

### Preparation of Polymer Particle P-4: Two Step Synthesis of Heavily Wrinkled Beads with Sulfonate Surface Groups

### Step 1: Synthesis of Sulfonated Polyester Stabilizer

TABLE 1

| | | | | Mole |
|---|---|---|---|---|
| Reagent | | Amount | | % in |
| # | Reagent | (g) | Mole | polymer |
| 1 | 5-sulfoisophthalic acid, | 47.96 | 0.41 | 50 |
| | dimethyl ester, sodium salt. | | | |
| 2 | 1,4-Cyclohexanedimethanol, | 119.17 | 0.83 | 100 |
| | mixture of cis/trans. | | | |
| 3 | Sodium acetate | 1.70 | 2.12 × 10⁻² | — |
| 4 | Zinc acetate dihydrate | 0.022 | 3.00 × 10⁻⁴ | — |
| 5 | Fascat 4100 | 0.018 | — | — |
| 6 | Fumaric acid | 47.96 | 0.41 | 50 |

Reagents 1-5, shown in Table 1, were combined in a 500 ml 3-neck flask equipped with a stainless steel stirring rod, nitrogen inlet, and an arm leading to a dry ice/acetone condenser connected to a graduated cylinder with a ground glass joint attached below the condenser to measure the collected condensate. The reaction was heated in bath containing a metal heating alloy. A steady stream of nitrogen was passed over the reaction mixture for 10 minutes, and then reduced to a slightly positive flow. The reaction was heated at 220° C. and slowly ramped to 250° C. over 460 minutes at which point a clear prepolymer had resulted and the expected amount of methanol had been collected. The reaction was removed from the heating bath and Reagent 6 was added. The reaction was then continued at 220° C. and within 10-15 minutes water condensate began to collect in the trap. The reaction was continued at 220° C. for 400 additional minutes until the polyester became too viscous to stir. The polyester was found to have Mn=2720 and Mw=6400 by size exclusion chromatography in dimethylformamide eluent.

### Step 2: Synthesis of Wrinkled polyester-Stabilized Particles

TABLE 2

| | Reagent | Amount (g) |
|---|---|---|
| 1 | Polyester of Step 1 | 20.0 |
| 2 | Water | 200.0 |
| 3 | Styrene | 10.7 |
| 4 | divinylbenzene | 2.7 |
| 5 | n-hexadecane | 1.3 |
| 6 | Toluene | 33.3 |
| 7 | Azobisisobutyronitrile | 0.7 |
| | (AIBN) | |

The sulfonated polyester stabilizer of Step 1 was heated in 750.0 ml water at ˜60° C. for 1 hour to afford a clear, slightly yellow solution, which was cooled to room temperature. An organic phase was prepared by combining Reagents 3-7, listed in Table 2. The polyester solution and the organic phase were combined in a 2 L beaker and mixed using a Silverson L4R mixer at the highest speed setting for 10 minutes. The resultant dispersion was poured into a 2 L, 3-neck round bottom flask fitted with a mechanical stirrer, reflux condenser, and nitrogen inlet and bubble degassed with nitrogen for 10 minutes. The reaction was then heated for 16 hours in a thermostatted water bath at 70° C. and the toluene was stripped as a water azeotrope using a rotary evaporator. The particle dispersion was purified by diafiltration with 4 volumes of water using a Millipore Amicon® ultrafiltration system with a 100K cutoff cartridge. The dispersion was freeze dried to yield a powder. The powder was redispersed in water using sonication for one minute to give a final dispersion of 20 wt. % solids. The mean particle size was determined to be 1.25 microns with a coefficient of variation of 53.60% using a Horiba LA-90 particle size analyzer. Analysis of the particles by electron microscopy (see FIG. 1) showed highly deformed spherical particles with deep ridges and wrinkles. Evaluation by Nitrogen BET gave a surface area of 28.29 m²/g.

### Preparation of Polymer Particle P-5: Two Step Synthesis of Heavily Wrinkled Beads with Sulfonate Surface Groups

### Step 1: Synthesis of Sulfonated Polyester Stabilizer

TABLE 3

| | | Amount | | Mole % in |
|---|---|---|---|---|
| Reagent # | Reagent | (g) | Mole | polymer |
| 1 | 5-sulfoisophthalic acid, | 32.93 | 0.11 | 8 |
| | dimethyl ester, sodium salt. | | | |
| 2 | Neopentyl glycol | 144.72 | 1.39 | 100 |
| 4 | Zinc acetate dihydrate | 0.084 | 3.8 × 10⁻⁴ | — |
| 5 | Fascat ® 4100 (tin catalyst | 0.088 | — | — |
| | manufactured by Atofina) | | | |
| 6 | Maleic anhydride | 125.36 | 1.28 | 92 |

Reagents 1-5, listed in Table 3, were combined in a 500 ml 3-neck flask equipped with a stainless steel stirring rod, nitrogen inlet, and an arm leading to a dry ice/acetone condenser connected to a graduated cylinder with a ground glass joint attached below the condenser to measure the collected condensate. The reaction was heated in a bath containing a metal heating alloy. A steady stream of nitrogen was passed over the reaction mixture for 10 minutes, and then reduced to a slightly positive flow. The reaction was heated at 220° C. and slowly ramped to 250° C. over 60 minutes, at which point a clear prepolymer had resulted and the expected amount of methanol had been collected. The heating bath was allowed to cool to 190° C. over 30 minutes and Reagent 6 was added. The reaction was then continued at 220° C. and within 10-15 minutes water condensate began to collect in the trap. The reaction was continued at 220° C. for 350 additional minutes and then terminated. The polyester was found to have Mn=3,510 and Mw=10,900 by size exclusion chromatography in dimethylformnarnide eluent.

### Step 2: Synthesis of Wrinkled Polyester-Stabilized Particles

TABLE 4

| | Reagent | Amount (g) |
|---|---|---|
| 1 | Polyester of Step 1 | 10.0 |
| 2 | Water | 428.57 |
| 3 | Styrene | 32.00 |
| 4 | divinylbenzene | 8.00 |
| 5 | n-hexadecane | 2.86 |
| 6 | Toluene | 92.86 |
| 7 | AIBN | 0.5 |

A procedure analogous to that described for Step 2 of Polymer Particle P-4 was conducted using the reagents listed above in Table 4 with the following changes: After removal of the toluene by rotary evaporation, the particle dispersion was further purified by three cycles of centrifugation, decantation of the clear supernatant, and redispersion in methanol. After the third decantation step, the damp solids were dried in a vacuum oven overnight at 80° C. to afford 31.45 g of a white powder. The powder was redispersed in water using sonication for one minute to give a final dispersion of 20 wt. % solids. The mean particle size was determined using a Horiba LA-90 particle size analyzer. The distribution was bimodal with major mode was present at 2.89 μm and a minor mode at 0.45 μm. Analysis of the particles by electron microscopy (see FIG. 2) showed spherical particles with wrinkled surfaces.

### Example 1

This Example illustrates the vinyl-sulfone containing bead with different binders and with different gelatin/particle ratios.

Preparation of Elements 1-4

A coating composition was prepared from 40.6 wt. % of dispersion P-1, 2.1 wt. % gelatin (acid processed osseine (APO) Code 4 gelatin), 0.16 wt. % bisvinylsulfonylmethane (BVSM) and 57.14 wt. % water, making the relative proportions of particles to gelatin 85/15 by weight. The solution was coated at various wet thicknesses onto a base support comprised of a polyethylene resin coated photographic paper stock, which had been previously subjected to corona discharge treatment, using a calibrated coating knife, and dried to remove substantially all solvent components to form a porous layer. The thickness of the dry layers was measured to be from about 5 to about 40±2 μm.

Preparation of Element 2

A coating composition was prepared from 39.0 wt. % of dispersion P-1, 3.8 wt. % gelatin (APO Code 4 gelatin), 0.3 wt. % BVSM and 56.9 wt. % water, making the relative proportions of particles to gelatin 75/25 by weight. The solution was coated and dried the same as Element 1. The thickness of the dry porous layer was measured to be about 30 ±2 μm.

Preparation of Element 3

A coating composition was prepared from 44.7 wt. % of dispersion P-1, 3.3 wt. % Witcobond W-320 dispersion (a 34.7 wt. % solids dispersion of polyurethane latex in water purchased from Witco Corporation), 1.11 wt. % gelatin (APO Code 4 gelatin), 0.09 wt. % BVSM and 50.8 wt. % water, making the relative proportions of particles to binder (gelatin +Witco 320) 85/15 by weight. The solution was coated and dried the same as Element 1. The thickness of the dry porous layer was measured to be about 38±2 μm.

Preparation of Element 4

A coating composition was prepared from 44.7 wt. % of dispersion P-1, 6.7 wt. % Witcobond W-320 dispersion (a 34.7 wt. % solids dispersion of polyurethane latex in water purchased from Witco Corporation), and 48.6 wt. % water, making the relative proportions of particles to binder Witco 320 85/15 by weight. The solution was coated and dried the same as Element 1. The thickness of the dry porous layer was measured to be about 40±2 μm.

### Example 2

Example 2 illustrates the effective use of various types of particles.

Preparation of Element 5

A coating composition was prepared from 59.0 wt. % of dispersion P-2, 2.3 wt. % gelatin (APO Code 4 gelatin), 0.18 wt. % BVSM and 38.52 wt. % water, making the relative proportions of particles to gelatin 85/15 by weight. The solution was coated and dried the same as Element 1. The thickness of the dry porous layer was measured to be about 30±2 μm.

Preparation of Element 6

A coating composition was prepared from 17.2 wt. % of dispersion P-4, 0.6 wt. % gelatin (APO Code 4 gelatin), 0.05 wt. % BVSM and 82.15 wt. % water, making the relative proportions of particles to gelatin 85/15 by weight. The solution was coated and dried the same as Element 1. The thickness of the dry porous layer was measured to be about 6±1 μm.

Preparation of Element 7

A coating composition was prepared from 17.2 wt. % of dispersion P-5, 0.6 wt. % gelatin (APO Code 4 gelatin), 0.05 wt. % BVSM and 82.15 wt. % water, making the relative proportions of particles to gelatin 85/15 by weight. The solution was coated and dried the same as Element 1. The thickness of the dry porous layer was measured to be about 5±1 μm.

### Example 3

Example 3 illustrates a porous polyester bead with different gelatin/particle ratios.

Preparation of Elements 8-12

Coating compositions were prepared from mixing dispersion P-3, with gelatin (APO Code 4 gelatin), BVSM and water in the amounts indicated in the Table below. The solutions were coated and dried the same as Element 1. The thickness' of the dry porous layers were all measured to be between about 3 and 4±1 μm.

TABLE 5

| Element | Wt. % P-3 | Wt. % gelatin | Wt. % BVSM | Wt. % water |
|---|---|---|---|---|
| 8 | 17.0 | 0.6 | 0.05 | 82.35 |
| 9 | 18.0 | 0.4 | 0.03 | 81.57 |
| 10 | 15.0 | 1.0 | 0.08 | 83.92 |
| 11 | 10.0 | 2.0 | 0.16 | 87.84 |
| 12 | 5.0 | 3.0 | 0.24 | 91.76 |

### Example 4

This example illustrates the method of evaluating a porous coated protein microarray support using a modified enzyme linked immunosobent assay (ELISA).

The procedure to perform the modified ELISA is follows.
1. Goat anti-mouse antibody IgG from Sigma was dissolved in PBS (phosphate saline buffer, pH7.4) buffer to a concentration of 1 mg/mL. A series of diluted of goat anti-mouse antibody IgG were spotted onto coated gelatin supports. The spotted supports were incubated in a humid chamber for 1 hour at room temperature.
2. The spotted supports were incubated in PBS buffer with 1% BSA for 1 hour with constant shaking.
3. The spotted supports were washed three times in PBS buffer with 0.05% Tween™ 20, 5 min each time with shaking.
4. Mouse IgG from Sigma was diluted in PBS buffer with 0.05% Tween™ 20 to 1 μg/mL to cover the whole surface of supports, and the supports were incubate at room temperature for 1 hour.
5. The supports were washed three times with PBS buffer with Tween™ 20, 5 min each time with constant shaking.
6. The supports were incubated in goat anti-mouse IgG horse raddish peroxidase conjugate (diluted in PBS with 1% glycine to appropriate titer) solution to cover the whole surface of the supports at room temperature for 1 hour with shaking.
7. The supports were washed three times with PBS buffer, 5 min each time with constant shaking, and rinsed twice in water.
8. The signals from the spots were generated using SuperSignal® ELISA chemiluminescence support solution (purchased from PIERCE ENDOGEN). The chemiluminescence image was capture by contacting a thin layer of SuperSignal® ELISA chemiluminescence support solution (purchased from PIERCE ENDOGEN) with coated support. The emission was measured on Kodak Image Station 440 and quantified using Region of Interest (ROI) software supplied with the instrument.

The results of evaluation on all coating elements are summarized below:

TABLE 6
Chemiluminescence intensity from ELISA
assay on various porous coatings

| | Goat anti-mouse | Goat anti-mouse | Goat anti-mouse |
|---|---|---|---|
| Element | IgG at 1 mg/mL | IgG at 0.2 mg/mL | IgG at 0.1 mg/mL |
| 1 | 2134 | 1362 | 640 |
| 2 | 1904 | 961 | 748 |
| 3 | 5197 | 3357 | 1397 |
| 4 | 5735 | 3798 | 1923 |
| 5 | 5173 | 2878 | 1798 |
| 6 | 4095 | 3844 | 3515 |
| 7 | 2984 | 2802 | 1940 |
| 8 | 188 | 21 | 12 |
| 9 | 79 | 58 | 8 |
| 10 | 187 | 121 | 78 |
| 11 | 390 | 251 | 194 |
| 12 | 1611 | 763 | 347 |

The invention has been described in detail with particular reference to certain preferred embodiments thereof, but it will be understood that variations and modifications can be effected within the spirit and scope of the invention.

1. A microarray comprising a support having attached to a surface thereof at least one porous layer, wherein said porous layer comprises a hydrophilic binder and polymer particles.

2. The microarray of claim 1 wherein said polymer particles comprise one or more polymers.

3. The microarray of claim 1 wherein said polymer particles comprise water insoluble synthetic polymers.

4. The microarray of claim 3 wherein said water insoluble synthetic polymers comprise at least one member selected from the group consisting of addition polymers, poly (alkylene oxides), phenol-formaldehyde polymers, urea-formaldehyde polymers and condensation polymers consisting of one or more of the following repetitive units: esters, amides, imides, carbonates, urethanes, and ethers.

5. The microarray of claim 1 wherein said polymer particles comprise monodisperse polymer particles.

6. The microarray of claim 5 wherein said monodisperse polymer particles have a particle size distribution, wherein the coefficient of variation of said particle size distribution is less than 20%.

7. The microarray of claim 5 wherein said monodisperse polymer particles have a particle size distribution, wherein the coefficient of said particle size distribution is less than 10%.

8. The microarray of claim 1 wherein said polymer particles comprise chemically active groups.

9. The microarray of claim 8 wherein said specific functionalities comprise chemically active groups present on the surface of said polymer particles.

10. The microarray of claim 8 wherein said chemically active groups comprise at least one member selected from the group consisting of thiols, primary amines, secondary amines, tertiary amines, quaternary ammoniums, phosphines, alcohols, carboxylic acids, primary or secondary amines, vinylsuflonyls, aldehydes, epoxies, hydrazides, succinimidyl esters, carbodiimides, maleimides, iodoacetyls, isocyanates, isothiocyanates, aziridines, or sulfonates.

11. The microarray of claim 8 wherein said chemically active groups comprise at least one member selected from the groups consisting of carboxylic acids, primary amines, secondary amines, or carboxylic acids.

12. The microarray of claim 8 wherein said chemically active groups comprise vinylsulfonyl units.

13. The microarray of claim 8 wherein said specific functionalities comprise chemically active groups present on stabilizer polymers which are covalently grafted, chemisorbed, or physically adsorbed to the surface of said polymer particles.

14. The microarray of claim 13 wherein said stabilizer polymers comprise at least one member selected from the groups consisting of poly(propyleneimine), polymers and copolymers of methacrylic acid, acrylic acid, mercaptomethyl styrene, N-aminopropyl (meth)acrylamide and secondary amine derivatives thereof, N-aminoethyl (meth)acrylate and secondary amine forms thereof, diallyamine, vinylbenzylamine, vinylamine, (meth)acrylic acid, vinylbenzyl mercaptan, and hydroxyethyl(meth)acrylate.

15. The microarray of claim 13 wherein said stabilizer polymers comprise at least one member selected from the groups consisting of poly(vinylamine), poly(propyleneimine), polyethyleneimine, polyacrylic acid, polymethacrylic acid, or poly(N-aminopropyl methacrylamide).

16. The microarray of claim 13 wherein said stabilizer polymers comprise pendant vinylsulfonyl or latent vinylsulfonyl groups.

17. The microarray of claim 16 wherein said stabilizer polymers are represented by Formula I: 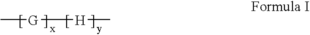
wherein
“G” represents a polymerized a,p-ethylenically unsaturated addition polymerizeable monomer;
“H” represents a vinylsulfone or vinylsulfone precursor unit monomer; and
x and y both represent molar percentages ranging from 10 to 90 and 90 to 10.

18. The microarray of claim 17 wherein x and y range from 25 to 75 and 75 to 25 respectively.

19. The microarray of claim 17 wherein G represents nonionic or ionic monomers.

20. The microarray of claim 19 wherein said ionic monomers comprise at least one member selected from the group consisting of 2-phosphatoethyl acrylate potassium salt, 3-phosphatopropyl methacrylate ammonium salt, acrylamide, methacrylamides, maleic acid and salts thereof, sulfopropyl acrylate and methacrylate, acrylic and methacrylic acids and salts thereof, N-vinylpyrrolidone, acrylic and methacrylic esters of alkylphosphonates, styrenics, acrylic and methacrylic monomers containing amine ammonium functionalities, styrenesulfonic acid and salts thereof, acrylic and methacrylic esters of alkylsulfonates, vinylsulfonic acid and salts thereof.

21. The microarray of claim 19 wherein said nonionic monomers comprise at least one member selected from the group consisting of poly(ethylene oxide) segments, carbohydrates, amines, amides, alcohols, polyols, nitrogen-containing heterocycles, and oligopeptides.

22. The microarray of claim 19 wherein said nonionic monomers comprise at least one member selected from the group consisting of poly(ethylene oxide) acrylate and methacrylate esters, vinylpyridines, hydroxyethyl acrylate, glycerol acrylate and methacrylate esters, (meth)acrylamide, and N-vinylpyrrolidone.

23. The microarray of claim 17 wherein G represents the polymerized form of acrylamide, sodium 2-acrylamido-2-methanepropionate, sulfopropyl acrylate and methacrylate salts, or sodium styrenesulfonate.

24. The microarray of claim 17 wherein H represents the polymerized form of a vinylsulfone or vinylsulfone precursor unit.

25. The microarray of claim 17 wherein said “H” represents groups represented by Formula II: 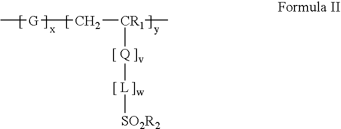
wherein:
R₁ is a hydrogen atom or a C₁-C₆ alkyl group. Preferably R₁ is a hydrogen atom.
Q is —CO₂—, or CONR₁.;
v is 1 or 0;
w is 1-3;
L is a divalent linking group containing at least one linkage selected from the group consisting of —CO₂— and —CONR₁, and containing 3-15 carbon atoms, or a divalent atom containing at least one linkage selected from the group consisting of —O—, —N(R₁)—, —CO—, —SO—, —SO₂—, —SO₃—, —SO₂N(R₁)—, —N(R₁)CON(R₁)— and —N(R₁)CO₂—, and containing 1-12 carbon atoms in which R₁ has the same meaning as defined above;
R₂ is —CH═CH2 or —CH2-CH2X₁ wherein X₁ is a substituent replaceable by a nucleophilic group or releasable in the form of HX₁ by a base.

26. The microarray of claim 25 wherein X₁ represents —S₂O₃⁻, —SO₄⁻, —Cl, —Br, —I, quaternary ammonium, pyridinium, and —CN, and sulfonate esters.

27. The microarray of claim 1 wherein said polymer particles comprise at least one ethylenically unsaturated polymerizable monomer.

28. The microarray of claim 27 wherein said at least one ethylenically unsaturated polymerizable monomer comprises at least one member selected from the group consisting of methacrylic acid esters, such as methyl methacrylate, ethyl methacrylate, isobutyl methacrylate, 2-ethylhexyl methacrylate, benzyl methacrylate, phenoxyethyl methacrylate, cyclohexyl methacrylate and glycidyl methacrylate, acrylate esters such as methyl acrylate, ethyl acrylate, isobutyl acrylate, 2-ethylhexyl acrylate, benzyl methacrylate, phenoxyethyl acrylate, cyclohexyl acrylate, and glycidyl acrylate, styrenics such as styrene, a-methylstyrene, 3- and 4-chloromethylstyrene, halogen-substituted styrenes, and alkyl-substituted styrenes, vinyl halides and vinylidene halides, N-alkylated acrylamides and methacrylamides, vinyl esters such as vinyl acetate and vinyl benzoate, vinyl ether, allyl alcohol and its ethers and esters, and unsaturated ketones and aldehydes such as acrolein and methyl vinyl ketone, isoprene, butadiene and acrylonitrile. Preferably, the monomers will be styrenics or acrylic esters or methacrylic esters.

29. The microarray of claim 27 wherein said at least one ethylenically unsaturated polymerizable monomer comprises chemical functionalities.

30. The microarray of claim 29 wherein said chemical functionalities comprise at least one member selected from the group consisting of vinyl groups, acrylates, methacrylates, vinyl ethers and vinyl esters.

31. The microarray of claim 27 wherein said at least one ethylenically unsaturated polymerizable monomer comprises trimethylolpropane triacrylate, ethylene glycol dimethacrylate, isomers of divinylbenzene, and ethylene glycol divinyl ether.

32. The microarray of claim 27 further comprising one or more water-soluble ethylenically unsaturated monomers, wherein said one or more water-soluble ethylenically unsaturated monomers comprises less than 20% of the total weight of said polymer particles.

33. The microarray of claim 32 wherein said one or more water-soluble ethylenically unsaturated monomers comprise at least one member selected from the groups consisting of styrenics, acrylates, and methacrylates substituted with highly polar groups, unsaturated carbon and heteroatom acids such as acrylic acid, methacrylic acid, fumaric acid, maleic acid, itaconic acid, vinylsulfonic acid, vinylphosphonic acid, and their salts, vinylcarbazole, vinylimidazole, vinylpyrrolidone, and vinylpyridines.

34. The microarray of claim 1 wherein said polymer particles have a mean diameter of from 0.05 to 50 microns.

35. The microarray of claim 1 wherein said polymer particles have a mean diameter of from 0.50 to 5 microns.

36. The microarray of claim 1 wherein said hydrophilic binder comprises at least one member selected from the groups consisting of gelatin, modified gelatin, water-soluble cellulose ethers, poly(n-isopropylacrylamide), polyvinylpyrrolidone and vinylpyrrolidone-containing copolymers, polyethyloxazoline and oxazoline-containing copolymers, imidazole-containing polymers, polyacrylamides and acrylamide-containing copolymers, poly(vinyl alcohol) and vinyl-alcohol-containing copolymers, poly(vinyl methyl ether), poly(vinyl ethyl ether), poly(ethylene oxide), acacia, alginic acid, bentonite, carbomer, carboxymethylcellulose sodium, cetostearyl alcohol, colloidal silicon dioxide, ethylcellulose, guar gum, hydroxyethylcellulose, hydroxypropyl cellulose, hydroxypropyl methylcellulose, magnesium aluminum silicate, maltodextrin, methylcellulose, povidone, propylene glycol alginate, sodium alginate, sodium starch glycolate, starch, tragacanth, xanthum gum, and mixtures thereof.

37. The microarray of claim 1 wherein said hydrophilic binder comprises gelatin.

38. The microarray of claim 37 wherein said gelatin comprises alkali pretreated gelatin.

39. The microarray of claim 1 wherein said hydrophilic binder comprises chemically active groups rich in specific functionalities.

40. The microarray of claim 39 wherein said specific functionalities comprise at least one member selected from the group consisting of thiols, primary amines, secondary amines, tertiary amines, phosphines, alcohols, carboxylic acids, vinylsulfonyls, aldehydes, epoxies, hydrazides, succinimidyl esters, carbodiimides, maleimides, iodoacetyls, isocyanates, isothiocyanates, or aziridines.

41. The microarray of claim 39 wherein said specific functionalities comprise at least one member selected from the group consisting of primary or secondary amines or a vinylsulfonyl group.

42. The microarray of claim 39 wherein said hydrophilic binder comprises at least one member selected from the group consisting of poly(propyleneimine), polymers and copolymers of N-aminopropyl (meth)acrylamide and secondary amine derivatives thereof, N-aminoethyl (meth)acrylate and secondary amine forms thereof, diallyamine, vinylbenzylamine, vinylamine, (meth)acrylic acid, vinylbenzyl mercaptan, and hydroxyethyl(meth)acrylate. Preferably, the polymer is poly(vinylamine), poly(propyleneimine), or poly(N-aminopropyl methacrylamide).

43. The microarray of claim 1 further comprising a bioaffinity tag bound to said at least one porous layer in a spatially addressable manner.

44. The microarray of claim 43 wherein said bioaffinity tag is bound to said hydrophilic binder of said at least one porous layer.

45. The microarray of claim 43 wherein said bioaffinity tag is bound to said polymer particle of said at least one porous layer.

46. The microarray of claim 43 wherein said bioaffinity tag is bound to said stabilizer polymer.

47. The microarray of claim 43 wherein said bioaffinity tag comprises at least one member selected from the group consisting of DNA, antibodies, antigens, proteins, enzymes, nucleic ligands, and polysaccharides.

48. The microarray of claim 1 wherein said at least one porous layer comprises from 0.25 to 250 microns in thickness.

49. The microarray of claim 1 wherein said at least one layer comprises more than a singlelayer to produce a three-dimensional array.

50. The microarray of claim 1 wherein said porous layer may also include crosslinking agents.

51. The microarray of claim 1 wherein said support comprises glass.

52. The microarray of claim 1 wherein said support comprises at least one member selected from the group consisting of glass, fused silica, plastics, metals, papers and semiconductors.

53. The microarray of claim 1 further comprising an under-coating or subbing layer between said porous layer and said support.

54. A method of using a microarray comprising:
providing a microarray comprising a support having attached to a surface thereof at least one porous layer, wherein said porous layer comprises a hydrophilic binder and polymer particles;
contacting said microarray with biological targets labeled with optical emission tag; and
measuring the signals from said optical emission tag.
